(19) United States
(12) Patent Application Publication
(10) Pub. No.: US 2022/0301065 A1
(43) Pub. Date: Sep. 22, 2022

(54) CLOUD EDITING SERVICE FOR INSURANCE CLAIMS

(71) Applicant: Change Healthcare Holdings, LLC, Nashville, TN (US)

(72) Inventors: Melinda Jennings, Salisbury, MD (US); Andrew Lauer, Oreland, PA (US); Ben Powell, Knoxville, TN (US); Sergey Polyakov, Norwalk, CT (US); Dede Ciprari, St. Paul, MN (US)

(21) Appl. No.: 17/203,367

(22) Filed: Mar. 16, 2021

(51) Int. Cl. G06Q 40/08; G16H 40/00; G16H 10/60

(52) U.S. Cl. CPC G06Q 40/08; G16H 40/00; G16H 10/60

(57) ABSTRACT

A method comprises receiving, at a first entity, an insurance claim for a health care service provided to a person by a provider; providing the claim from the first entity to an editing service, wherein the editing service is configured to edit the claim in accordance with information associated with a second entity and generate an edited claim, wherein the second entity is different than the first entity; receiving, at the first entity, the edited claim from the editing service; and determining, at the first entity, a payment to be made to the provider based on the edited claim. This method may be performed during the claim's adjudication cycle in realtime, not retrospectively.

### BACKGROUND

A person who belongs to an insurance plan provided by an insurance company is referred to as a member. The insurance plan often has a geographic home service area, such as a home state or home region, and is referred to as a “home plan” that “owns” the member. Thus, a home plan is the plan where the insured (i.e., the member) is enrolled.

Health care services are delivered to members through health care providers, e.g., one or more medical practitioners. Payment for these services is usually made by one or more payors, which may include the member and another entity, such as an insurance company. Payors (e.g., insurance companies) may be harmed financially for approving payment for claims that cannot be justified. Often, payors use rules that can be applied to the claims to make the recommendations on payment.

A member may be outside of their home plan's home service area, and in another geographic service area referred to as a host area (e.g., a host state or host region). Such a member may be referred to as an “out-of-area insured”. An insurance company in the host area will have its own insurance plan that has its own rules, contracts, and historical claim information for paying claims and providers, which is often different than other insurance plans such as the home plan. The insurance plan in the host area is referred to herein as a “host plan” with respect to the member who is visiting or otherwise present from outside their home plan's home service area. Thus, a host plan is the plan serving the area where the actual medical service was provided to the member.

When a member files a claim in the host area (e.g., because they have received care from a provider in the host area), the claim is filed in accordance with the host plan's rules and policies, which may not be the same as the home plan's rules and policies. The host plan insurance company then pays the provider of the member's claim, and then the host plan insurance company seeks reimbursement from the home plan insurance company. Such post-pay recovery is inefficient and often results in a lack of end-to-end payment integrity as well as economic loss (e.g., lost time, improperly spent money, higher costs, etc.) for the home plan insurance company, the host plan insurance company, or both. It is noted that recovery payments typically only happen for claims that are at risk for overpayment (e.g., those where the member history claim payment is not available).

As an example, if a member of a Michigan home plan is injured in Florida and receives treatment from a provider in Florida, the member (or the provider) submits an insurance claim to the host plan (the insurance plan of the Florida insurance company). The Florida insurance company will process the claim under its rules and using its data, and will pay the provider. The home plan insurance company (the Michigan insurance company) will reimburse the Florida insurance company. However, the home plan insurance company may determine that the provider should have been paid a different amount than the host plan insurance company had determined and paid to the provider. The home plan insurance company may make this determination using its home insurance plan rules and its historical claim data. The difference between the amount of money that the host plan insurance company paid the provider, and the amount of money that the home plan insurance company determines is the proper amount that should have been paid to the provider must then be rectified.

It is with respect to these and other considerations that the various aspects and embodiments of the present disclosure are presented.

### SUMMARY

The systems and methods described herein provide for a claim filed in a host area in accordance with a host plan to be analyzed (e.g., by an editing service such as a cloud editing service) against the home plan of the home area, including against the home plan's rules, policies, and/or past claims. Some aspects also provide suggested revisions to the submitter (the entity that submitted the claim to the host plan).

In an implementation, a method comprises: receiving, at a first entity, a claim for a service provided to a person by a provider; providing the claim from the first entity to an editing service, wherein the editing service is configured to edit the claim in accordance with information associated with a second entity and generate an edited claim, wherein the second entity is different than the first entity; receiving, at the first entity, the edited claim from the editing service; and determining, at the first entity, a payment to be made to the provider based on the edited claim.

In an implementation, a method comprises: receiving, at an editing service, a claim for a service provided to a person by a provider; generating an edited claim, at the editing service, using rules of a first entity and information of a second entity, wherein the rules of the first entity align with contracts of the provider, wherein the second entity is different than the first entity; and providing the edited claim from the editing service to the first entity.

In an implementation, a method comprises: receiving, at a first entity, a claim for a service provided to a person by a provider; applying rules and edits of the first entity to the claim, by the first entity; determining, at the first entity, that the claim is for a member of a plan outside of the plan of the first entity, wherein the plan is of a second entity, wherein the second entity is different than the first entity; providing the claim from the first entity to an editing service, wherein the editing service is configured to edit the claim in accordance with information associated with a second entity and generate an edited claim; receiving, at the first entity, the edited claim from the editing service; and determining, at the first entity, a payment to be made to the provider based on the edited claim.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is an illustration of an exemplary environment for editing insurance claims;

FIG. 2 is a block diagram of an implementation of an editing service;

FIG. 3 is a block diagram of another implementation of an editing service;

FIG. 4 is an operational flow of an implementation of a method of editing insurance claims;

FIG. 5 is an operational flow of another implementation of a method of editing insurance claims;

FIG. 6 is an operational flow of another implementation of a method of editing insurance claims; and

FIG. 7 shows an exemplary computing environment in which example embodiments and aspects may be implemented.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components, and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care services to a patient.

Some embodiments of the inventive concept are described with reference to a payor determining whether to make payment for a claim for services or products. In the context of a health care environment, a payor may be, for example, an entity that provides health or medical insurance, such as private insurance companies and government insurance agencies, both at the federal and state levels (e.g., Medicare, Medicaid, public employee insurance agencies, and the like). Health care providers may submit claims for medical services rendered, products prescribed (e.g., medications, medical devices, etc.), administrative fees, and/or other fees or expenses to a payor for payment. Upon receiving a claim, the payor may then determine whether to pay the claim in whole, in part, or deny the claim. A claim may include both header and line information. The header may be information that applies to the entire claim, e.g., patient details (name, date of birth, address, etc.). The line information may identify the various services, products, fees, expenses, and/or other items for which payment is sought and may be referred to as line item(s). Thus, a payor may evaluate each line in the claim to determine whether to pay the invoiced amount in full, in part, or to deny payment for that line.

A payor, such as an insurance company operating in the health care field, may make use of a claims auditing system to assist them in determining whether to pay and how much to pay for the various lines listed in claims submitted for payment. Payors may be penalized for improperly denying payment of a claim. But a payor may suffer economically if payment is made for fraudulent claims or claims for which reimbursement has not been authorized or contracted for. Oftentimes, in order for proper payment accuracy, payors benefit from insight (e.g., information) regarding a member's claims history.

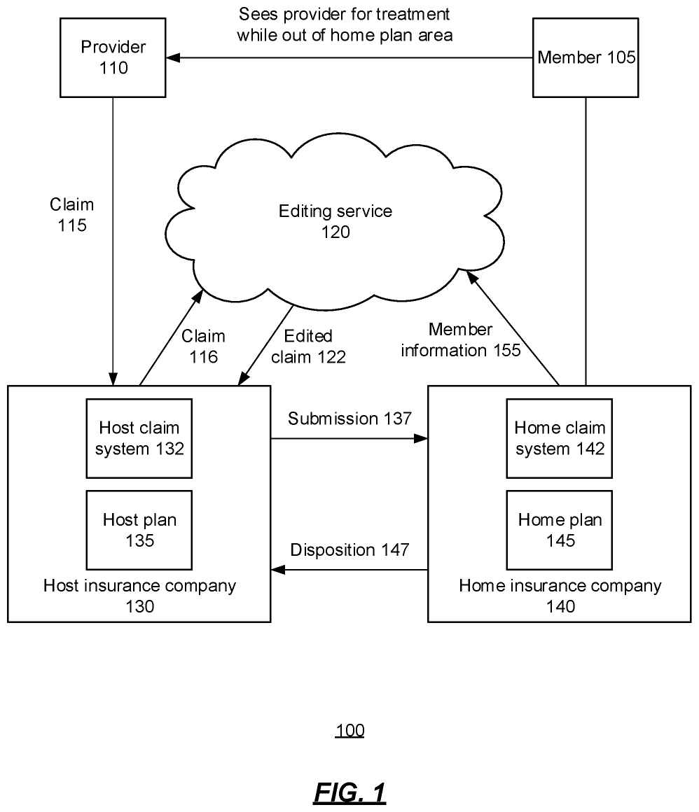

FIG. 1 is an illustration of an exemplary environment 100 for editing insurance claims. A member 105 is shown along with a provider 110. The member 105 is a member (i.e., an insured person) of a home insurance company 140. The home insurance company 140 comprises a home claim system 142 and a home plan 145 (i.e., a home insurance plan that covers the member 105). A host insurance company 130 is shown and comprises a host claim system 132 and a host plan 135 (a host insurance plan that covers members (not shown) of the host insurance company 130).

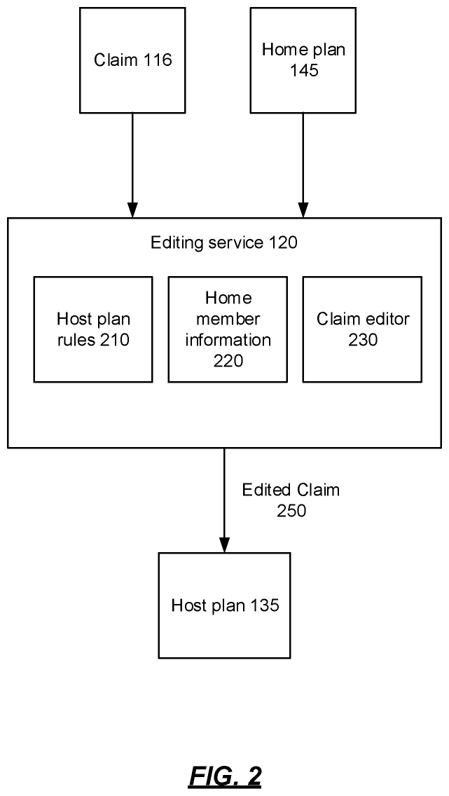

An editing service 120 is provided, e.g., in a cloud (i.e., as a cloud service) although this is not intended to be limiting, as the editing service 120 can reside or be comprised within one or more computing devices not in a cloud or otherwise outside a cloud, such as within the host insurance company 130, the home insurance company 140, and/or as a standalone system or service. The editing service 120 is described further herein, e.g., with respect to FIGS. 2-7. As is well known, cloud computing is the delivery of computing services—including servers, storage, databases, networking, software, analytics, and intelligence—over the Internet (“the cloud”). The term “cloud” as used herein is consistent with this definition.

One or more of the member 105, the provider 110, the editing service 120, the host insurance company 130, and the home insurance company 140 (e.g., one or more computing devices associated therewith) may be in communication through a network. The network may be a variety of network types including a packet switched network (e.g., the Internet) and private network communication circuits, for example.

Although only one host insurance company 130, one host claim system 132, one host plan 135, one home insurance company 140, one home claim system 142, one home plan, and one editing service 120 are shown, this is not intended to be limiting. Any number of host insurance companies 130, host claim systems 132, host plans 135, home insurance companies 140, home claim systems 142, home plans 145, and editing services 120 are contemplated and may be supported depending on the implementation. Similarly, any number of members 105 and providers 110 may be incorporated or used in the environment 100, depending on the implementation.

Here, the member 105 is out of the home plan area (and in the host plan area) and sees or otherwise engages the provider 110 for treatment (e.g., of a medical condition). The provider 110 provides treatment to the member 105, and submits a claim 115 to the host insurance company 130. The claim may be institutional or professional, inpatient or outpatient, in-network or out-of-network, for example.

The host insurance company 130 (e.g., the host claim system 132) receives and processes the claim 115 in accordance with the host plan 135 and the editing service 120, as described further herein. More particularly, the host claim system 132 sends a claim 116 to the editing service 120 for generation of an edited claim 122. The editing service 120 provides the edited claim 122 to the host insurance company 130. The claim 116 that is provided from the host insurance company 130 to the editing service 120 may be the original claim 115 or may be a claim based on the original claim 115, such as a claim that has received a first set of edits by the host insurance company 130 (e.g., by the host claim system 132). Editing the claim 116 does not require an actual change to the claim 116. In some implementations, editing the claim 116 results in the generation of a separate claim (or document, file, etc.) referred to herein, for example, as the edited claim 122. In some implementations, editing the claim 116 may include analysis and/or review of the claim 116 without an actual change to the claim 116. It is contemplated that when the editing service 120 applies rule logic to claim lines, some claim lines will pass and should be paid; some claim lines may be denied and not paid at all; and some claim lines may have a recommendation for reduced payment. Some claims may have all lines pass with no edit recommendations. Other claims may have several claim lines not recommended for payment.

The host insurance company 130 may pay the provider 110 for the claim 115 (e.g., pay all, part, or none of the claim 115), and provide a submission 137 to the home insurance company 140 for reimbursement and/or other information. The home insurance company 140 provides a disposition 147 (e.g., a reimbursement and/or other information) to the host insurance company 130.

In some implementations, a call may be made to the cloud for editing (e.g., secondary editing) of an out-of-plan (e.g., out-of-area insured) claim (i.e., a host claim). In some implementations, the Interplan Telecommunications System (ITS) may be used to allow a contracting provider to submit claims for out-of-area insureds to the provider's local plan (the host plan) for reimbursement. The ITS is well known and may be used to handle the interchange in some implementations. The member may have an identification number that contains an indicator (e.g., a three character alpha indicator or prefix) that can be used to identify claims that can be processed using the editing service 120 described herein.

In some implementations, the cloud may comprise an operational data store (ODS), a database that stores historical member claims (e.g., member information and/or member claim information, referred to herein as member information such as the member information 155) from the home plan. An ODS is a database that provides the latest member data. Claim edits performed herein use historical claim information (e.g., whether or not the claim is likely to be accepted/denied/reimbursed only partially, information in previous claims submitted by the member), as well as information in the claim itself, to determine or generate an edited claim.

In some implementations, the editing service 120 may comprise one or more rules engines (e.g., in the cloud) that have rules configured per provider contracts. For example, there may be one rules engine for each host plan and/or for each home plan, depending on the implementation.

The editing service 120 uses the home member data (e.g., the member information 155 provided to the editing service 120 by the home insurance company 140, the home plan rules, the home plan edits, etc. depending on the implementation) from the ODS, as well as the rules and edits from the host plan, to perform robust editing of the claim in a prepay fashion (i.e., prior to making any payment to the provider). This results in substantial cost savings because member claim paid history is utilized in the claim editing at this stage. In other words, substantial cost savings are realized because member history is used on host claims during adjudication (e.g., during the claim's adjudication cycle in realtime, not retrospectively). This will also increase provider satisfaction because providers do not like the “pay and chase” recovery method that otherwise would be used to try to recover on missed editing. Without the context of home member data (e.g., member claim history) as provided herein, common claim edits such as procedure-to-procedure bundling edits, claim line service edit denials, frequency edits, and multiple procedure payment reduction recommendations can be missed, resulting in a high probability of overpayments. The embodiments described herein reduce or eliminate post-pay overpayment audits. Such post-pay overpayment audits include increased administrative expense and create provider abrasion.

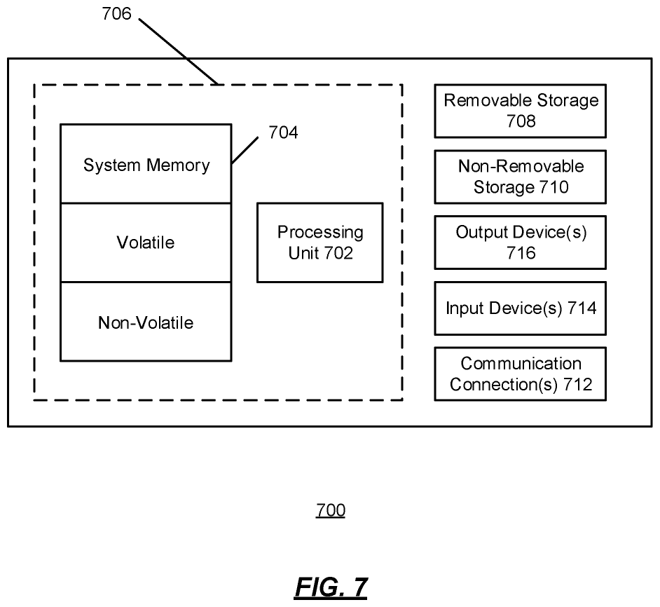

The host claim system 132 and the home claim system 142 may be configured to receive claims and coordinate payment for claims (e.g., generated by providers such as health care or medical service providers). The claim systems 132, 142 may use different formats, but the claims may each include header information and line information or line items as known in the art. The payor (e.g., the respective insurance company) may use the associated claim system (alone or in conjunction with the editing service 120 and/or other known data centers, data service, and data providers) to organize the claims and evaluate whether to pay the claims in whole, in part, or to deny the claims. Each claim system 132, 142 may be implemented using a variety of computing devices such as desktop computers, laptop computers, and the like Other types of computing devices may be supported. A suitable computing device is illustrated in FIG. 7 as the computing device 700. Cloud-based implementations are also contemplated.

FIG. 2 is a block diagram of an implementation of an editing service, such as the editing service 120. The editing service 120 comprises a plurality of modules, such as modules that comprise host plan rules 210, home member information 220, and a claim editor 230.

The editing service 120 receives the claim 116 of the provider 110 (e.g., from the host plan 135) with respect to the treatment of the member 105 and also receives information from the home plan 145 (e.g., the home member information 220 which may be based on or comprise the member information 155). The claim editor 230 uses the host plan rules 210 and the home member information 220 (e.g., the member information 155 provided to the editing service 120 by the home insurance company 140, the home plan rules, the home plan edits, etc. depending on the implementation) to generate an edited claim 250. Such editing at this stage avoids post-pay (i.e., post-provider reimbursement) corrections.

The host plan rules 210 may contain rules directed to medical guidelines, fraud, waste, etc., for example. The host plan rules 210 may be comprised within one or more rules engines that have rules configured per provider contracts, in some implementations.

In some implementations, the home member information 220 comprises historical patient claims, historical member claims, and/or home plan rules. The home member information may be stored on the cloud (e.g., in one or more ODSes) and/or in other storage accessible to the editing service 120, the host insurance company 130 including the host claim system 132 and the host plan 135, and/or the home insurance company 140 including the home claim system 142 and the home plan 145. There may be a daily (or other periodic or time-based) feed of member paid claims (e.g., as at least part of the member information 155) from the home plan to the stored home member information 220.

The editing service 120 provides the edited claim 250 to the host plan 135. The host insurance company 130 may then use the edited claim 250 to make a payment decision (e.g., a payment) to the provider 110 with respect to the claim 115 initially submitted by the provider 110 to the host insurance company 130 (via the host claim system 132, for example).

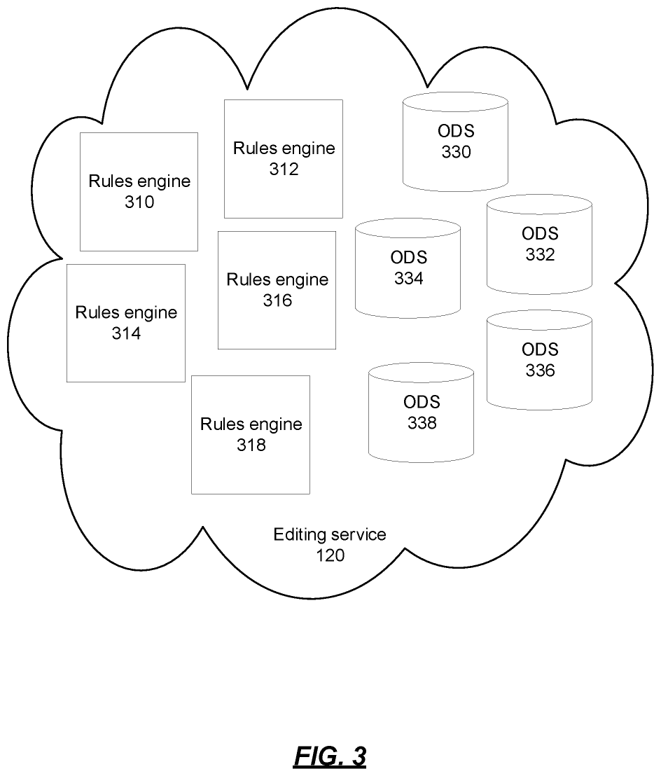

FIG. 3 is a block diagram of another implementation of an editing service, such as the editing service 120. Here, the editing service 120 is deployed as a cloud service, and comprises a plurality of rules engines 310, 312, 314, 316, 318, and a plurality of ODSes 330, 332, 334, 336, 338. Although five rules engines and five ODSes are shown, this is not intended to be limiting, as any number of rules engines and ODSes may be comprised within, or used in conjunction with, the editing service 120. Depending on the implementation, each host plan and/or each home plan may be affiliated with a respective one of the rules engines and/or one of the ODSes.

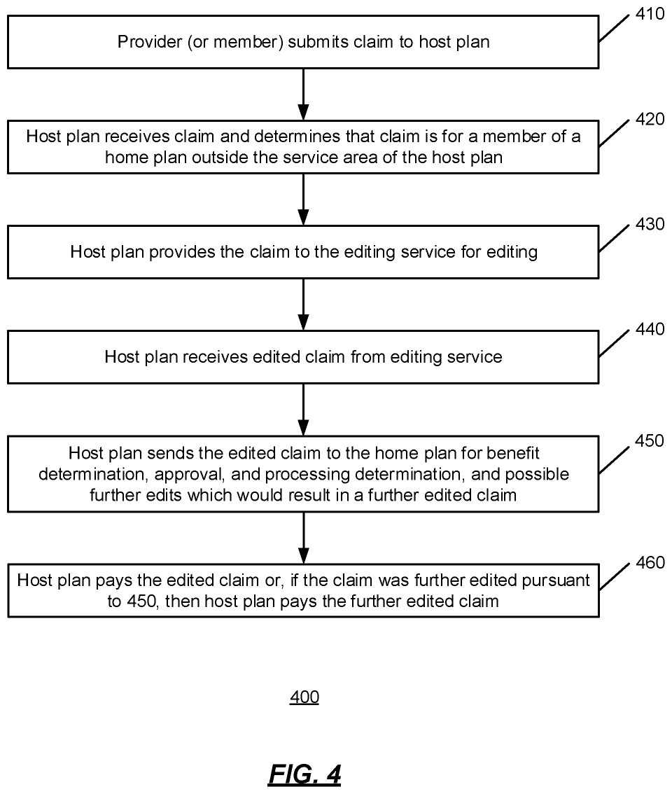

FIG. 4 is an operational flow of an implementation of a method 400 of editing insurance claims.

At 410, a provider or a member (or other entity) submits a claim, such as the claim 115, to a host plan. The submission may be performed using any known technique, and may be received by the host claim system 132 of the host insurance company 130.

At 420, the host plan (e.g., the host claim system 132 associated with the host plan 135) receives the claim and determines that the claim is for a member of a home plan (such as the home plan 145) outside the service area of the host plan. This may be determined by an ITS indicator of Yes or No, in some implementations, where a Yes indicator indicates that the claim is to be sent to the editing service (e.g., sent to the cloud for editing by the editing service 120). In some implementations, the three character prefix at the beginning of the member identification number is the key element used to identify and properly route the claims, and to ensure that the proper member history is retrieved from the proper ODS. The three character prefix may identify the plan to which the member belongs and may be used to confirm the member's membership and coverage.

At 430, when it is determined (from 420) that the claim is to be sent to an editing service, the host plan provides the claim to an editing service, such as the editing service 120, for editing and generation of an edited claim such as the edited claim 122 or the edited claim 250. Any known technique(s) for calling the editing service and/or providing the claim to the editing service may be used.

At 440, the host plan receives the edited claim from the editing service.

At 450, after editing, the host plan sends the edited claim to the home plan for benefit determination, approval, and processing determination. The home plan may provide and/or suggest further edits to the received edited claim for the host plan to consider and/or implement to potentially generate a further edited claim. Alternatively or additionally, the home plan may generate a further edited claim and provide the further edited claim to the host plan.

At 460, the host plan pays the provider for the claim based on information in the edited claim and/or the further edited claim from 440 and/or 450. The payment may be for all, some, or none of the original claim (i.e., payment, payment in part, or non-payment).

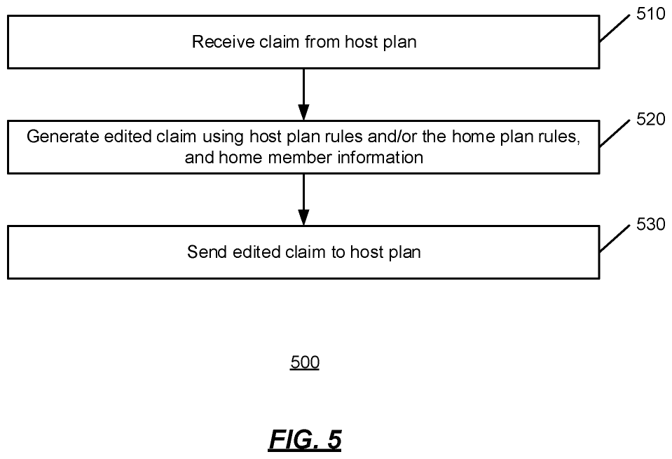

FIG. 5 is an operational flow of another implementation of a method 500 of editing insurance claims. The method may be performed by an editing service, such as the editing service 120.

At 510, a claim from a host plan is received at an editing service, such as the editing service 120. The editing service identifies the home plan based on the member ID, and obtains the historical member claim information from the appropriate data store (e.g., the ODS associated with the home plan of the member)

At 520, the editing service generates an edited claim using the host plan rules and/or the home plan rules (depending on the implementation), and the home member information.

At 530, the editing service sends the edited claim to the host plan (e.g., back to the host insurance company). Alternatively or additionally, information pertaining to the claim (e.g., the originally received claim from 510 and/or the edited claim) may be provided from the editing service to the host plan (e.g., likely denied, reimbursed, reimbursed in part).

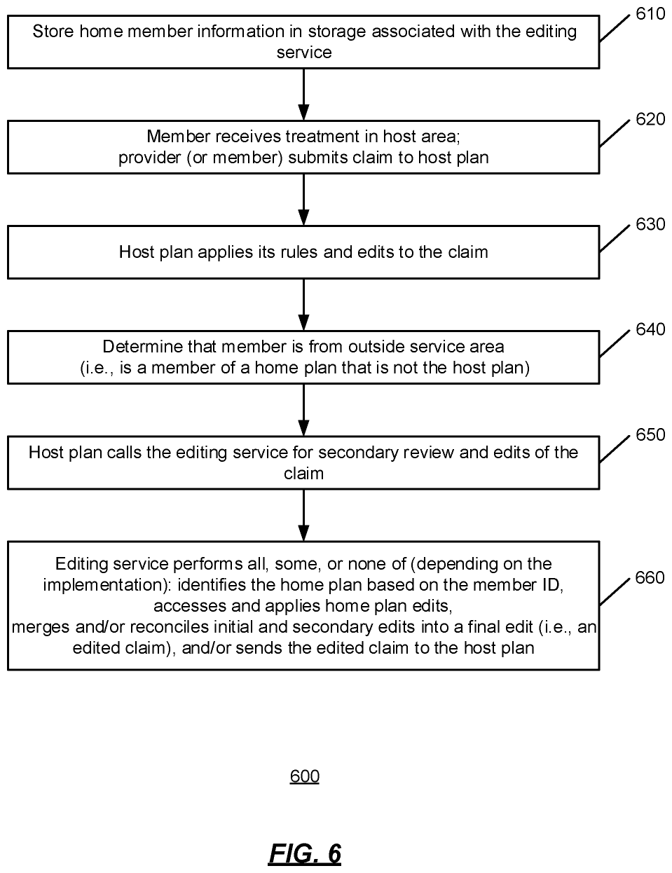

FIG. 6 is an operational flow of another implementation of a method of editing insurance claims.

At 610, home member information, such as the home member information 220, is stored in storage associated with an editing service, such as the editing service 120. The home member information may be stored in an ODS of the editing service, where the ODS is affiliated with the home plan. The home insurance company may control how much or how little claim history and/or member information is provided to the storage (e.g., the ODS) as the home member information. In some embodiments, the home insurance company may elect to only communicate historical claim information associated with paid claims because a paid claim is typically a final decision which will lead to more accurate claim editing.

At 620, a member of the home plan receives treatment from a provider in a host area. The provider (or member or other entity seeking payment or reimbursement) submits a claim to the host plan (e.g., using the standard process to submit a claim).

At 630, the host plan applies its rules and edits (e.g., applies contract and clinical edits) to the claim, e.g., using a claim editor, such as the claim editor 230. This may be considered to be a first set of edits to the claim. Depending on the implementation, at least some of the edits may be to align the claim with the contract(s). such that the edited claim is consistent with the associated contract(s) and meets the contractual agreement(s) and/or obligation(s).

At 640, the host plan determines that the member is from outside the service area of the host plan (i.e., the member is a member of a home plan that is not the host plan). In some implementations, the claim may have an indicator (e.g., an ITS indicator of Yes or No) to indicate whether or not the claim is from a member outside the service area and thus should be sent to the editing service 120. In some implementations, information from the member ID (e.g., the first three characters of the member ID) identifies which home plan the member is affiliated with, and thus can be used to determine whether or not to send the claim to the editing service 120 (and which rules engine and/or ODS to use, depending on the implementation).

At 650, the host plan calls the editing service for a secondary review of the claim and/or edits to the claim.

Depending on the implementation, at 660, the editing service may perform all, some, or none of the following: identifies the home plan based on the member identification or identifier, accesses and applies home plan edits (i.e., secondary edits or a second set of edits to the claim), merges and/or reconciles the initial edits (the first set of edits from 630) and secondary edits (the second set of edits), and sends the edited claim to the host plan. At this point, the host plan can use the edited claim to determine a payment, if any, to make to the provider (or other entity that submitted the claim at 620).

FIG. 7 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 7, an exemplary system for implementing aspects described herein includes a computing device, such as computing device 700. In its most basic configuration, computing device 700 typically includes at least one processing unit 702 and memory 704. Depending on the exact configuration and type of computing device, memory 704 may be volatile (such as random access memory (RAM)), non-volatile (such as read-only memory (ROM), flash memory, etc.), or some combination of the two. This most basic configuration is illustrated in FIG. 7 by dashed line 706.

Computing device 700 may have additional features/functionality. For example, computing device 700 may include additional storage (removable and/or non-removable) including, but not limited to, magnetic or optical disks or tape. Such additional storage is illustrated in FIG. 7 by removable storage 708 and non-removable storage 710.

Computing device 700 typically includes a variety of computer readable media. Computer readable media can be any available media that can be accessed by the device 700 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 704, removable storage 708, and non-removable storage 710 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 700. Any such computer storage media may be part of computing device 700.

Computing device 700 may contain communication connection(s) 712 that allow the device to communicate with other devices. Computing device 700 may also have input device(s) 714 such as a keyboard, mouse, pen, voice input device, touch input device, etc. Output device(s) 716 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

In an implementation, a method is provided. The method includes receiving, at a first entity, a claim for a service provided to a person by a provider; providing the claim from the first entity to an editing service, wherein the editing service is configured to edit the claim in accordance with information associated with a second entity and generate an edited claim, wherein the second entity is different than the first entity; receiving, at the first entity, the edited claim from the editing service; and determining, at the first entity, a payment to be made to the provider based on the edited claim.

Implementations may include some or all of the following features. The first entity is a first insurance company, the second entity is a second insurance company, and the claim is an insurance claim, wherein the person is a member of the second insurance company and is not a member of the first insurance company, wherein the first insurance company has contracts with the provider. The first entity is a host insurance company, the second entity is a home insurance company, and the claim is an insurance claim for a health care service provided to the person, wherein the person is a member of the home insurance company and is not a member of the host insurance company. The editing service is a cloud editing service. The method further comprises determining that the claim is for a member of a plan outside of the plan of the first entity, and only providing the claim from the first entity to the editing service after determining that the claim is for the member of the plan outside of the plan of the first entity. The method further comprises determining that the claim is for a member of a plan of the second entity. The determining is performed by checking an identifier of the member associated with the claim. The method further comprises making the payment from the first entity to the provider.

In an implementation, a method is provided. The method includes receiving, at an editing service, a claim for a service provided to a person by a provider; generating an edited claim, at the editing service, using rules of a first entity and information of a second entity, wherein the rules of the first entity align with contracts of the provider, wherein the second entity is different than the first entity; and providing the edited claim from the editing service to the first entity.

Implementations may include some or all of the following features. The claim is received at the editing service from the first entity. The editing service is a cloud editing service. The first entity is a first insurance company, the second entity is a second insurance company, and the claim is an insurance claim, wherein the person is a member of the second insurance company and is not a member of the first insurance company. The first entity is a host insurance company, the second entity is a home insurance company, and the claim is an insurance claim for a health care service provided to the person, wherein the person is a member of the home insurance company and is not a member of the host insurance company. The rules of the first entity comprise at least one of rules configured per provider contracts or rules directed to medical guidelines, and wherein the information of the second entity comprises at least one of historical claim information or rules of the second entity. Generating the edited claim comprises: identifying a plan based on a member identifier; applying edits of the plan to the claim; and merging or reconciling any previous edits to the claim into a final edit.

In an implementation, a method is provided. The method includes receiving, at a first entity, a claim for a service provided to a person by a provider; applying rules and edits of the first entity to the claim, by the first entity; determining, at the first entity, that the claim is for a member of a plan outside of the plan of the first entity, wherein the plan is of a second entity, wherein the second entity is different than the first entity; providing the claim from the first entity to an editing service, wherein the editing service is configured to edit the claim in accordance with information associated with a second entity and generate an edited claim; receiving, at the first entity, the edited claim from the editing service; and determining, at the first entity, a payment to be made to the provider based on the edited claim.

Implementations may include some or all of the following features. The editing service is further configured to edit the claim in accordance with information associated with the first entity. The information associated with the first entity comprises at least one of rules configured per provider contracts or rules directed to medical guidelines, and wherein the information of the second entity comprises at least one of historical claim information or rules of the second entity. The first entity is a host insurance company, the second entity is a home insurance company, and the claim is an insurance claim for a health care service provided to the person, wherein the person is a member of the home insurance company and is not a member of the host insurance company, and wherein the editing service is a cloud editing service, and further comprising making the payment from the first entity to the provider. Determining that the claim is for a member of a plan outside of the plan of the first entity, comprises checking an identifier of the member associated with the claim.

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method comprising:
receiving, at a first entity, a claim for a service provided to a person by a provider;
providing the claim from the first entity to an editing service, wherein the editing service is configured to edit the claim in accordance with information associated with a second entity and generate an edited claim, wherein the second entity is different than the first entity, and wherein the editing service edits the claim by:
accessing, via a cloud-based rules engine associated with the first entity, rules that align with contracts between the first entity and the provider;
retrieving, from a cloud-based operational data store (ODS) associated with the second entity, an electronic medical claims history for the person, wherein the cloud-based ODS is periodically updated with a feed of electronic medical claims for the person; and
modifying the claim according to the rules of the first entity and the retrieved electronic medical claims history for the person;
receiving, at the first entity, the edited claim from the editing service; and
determining, at the first entity, a payment to be made to the provider based on the edited claim.

2. The method of claim 1, wherein the first entity is a first insurance company, the second entity is a second insurance company, and the claim is an insurance claim, wherein the person is a member of the second insurance company and is not a member of the first insurance company, wherein the first insurance company has contracts with the provider.

3. The method of claim 1, wherein the first entity is a host insurance company, the second entity is a home insurance company, and the claim is an insurance claim for a health care service provided to the person, wherein the person is a member of the home insurance company and is not a member of the host insurance company.

4. The method of claim 1, wherein the editing service is a cloud editing service.

5. The method of claim 1, further comprising determining that the claim is for a member of a plan outside of the plan of the first entity, and only providing the claim from the first entity to the editing service after determining that the claim is for the member of the plan outside of the plan of the first entity.

6. The method of claim 5, further comprising determining that the claim is for a member of a plan of the second entity.

7. The method of claim 5, wherein the determining is performed by checking an identifier of the member associated with the claim.

8. The method of claim 1, further comprising making the payment from the first entity to the provider.

9. A method comprising:
receiving, at an editing service, a claim for a service provided to a person by a provider;
accessing, via a cloud-based rules engine associated with a first entity, rules that align with contracts between the first entity and the provider;
retrieving, from a cloud-based operational data store (ODS) associated with a second entity that is different from the first entity, an electronic medical claims history for the person, wherein the cloud-based ODS is periodically updated with a feed of electronic medical claims for the person;
generating an edited claim, at the editing service, by modifying the claim according to the rules of the first entity and the retrieved electronic medical claims history for the person; and
providing the edited claim from the editing service to the first entity.

10. The method of claim 9, wherein the claim is received at the editing service from the first entity.

11. The method of claim 9, wherein the editing service is a cloud editing service.

12. The method of claim 9, wherein the first entity is a first insurance company, the second entity is a second insurance company, and the claim is an insurance claim, wherein the person is a member of the second insurance company and is not a member of the first insurance company.

13. The method of claim 9, wherein the first entity is a host insurance company, the second entity is a home insurance company, and the claim is an insurance claim for a health care service provided to the person, wherein the person is a member of the home insurance company and is not a member of the host insurance company.

14. The method of claim 9, wherein the rules of the first entity comprise at least one of rules configured per provider contracts or rules directed to medical guidelines, and wherein the information of the second entity comprises at least one of historical claim information or rules of the second entity.

15. The method of claim 9, wherein generating the edited claim comprises:
identifying a plan based on a member identifier;
applying edits of the plan to the claim; and
merging or reconciling any previous edits to the claim into a final edit.

16. A method comprising:
receiving, at a first entity, a claim for a service provided to a person by a provider;
modifying the claim a first time by applying rules and edits of the first entity to the claim, by the first entity;
determining, at the first entity, that the claim is for a member of a plan outside of the plan of the first entity, wherein the plan is of a second entity, wherein the second entity is different than the first entity;
providing the claim from the first entity to an editing service, wherein the editing service is configured to edit the claim and generate an edited claim by:
retrieving, from a cloud-based operational data store (ODS) associated with a second entity that is different from the first entity, an electronic medical claims history for the person, wherein the cloud-based ODS is periodically updated with a feed of electronic medical claims for the person; and
modifying the claim a second time based on the retrieved electronic medical claims history for the person;
receiving, at the first entity, the edited claim from the editing service; and
determining, at the first entity, a payment to be made to the provider based on the edited claim.

17. The method of claim 16, wherein the editing service is further configured to edit the claim in accordance with information associated with the first entity.

18. The method of claim 17, wherein the information associated with the first entity comprises at least one of rules configured per provider contracts or rules directed to medical guidelines, and wherein the information of the second entity comprises at least one of historical claim information or rules of the second entity.

19. The method of claim 16, wherein the first entity is a host insurance company, the second entity is a home insurance company, and the claim is an insurance claim for a health care service provided to the person, wherein the person is a member of the home insurance company and is not a member of the host insurance company, and wherein the editing service is a cloud editing service, and further comprising making the payment from the first entity to the provider.

20. The method of claim 16, wherein determining that the claim is for a member of a plan outside of the plan of the first entity, comprises checking an identifier of the member associated with the claim.
